(19) United States
(12) Patent Application Publication
(10) Pub. No.: US 2009/0234220 A1
(43) Pub. Date: Sep. 17, 2009

(54) Catheter and associated medical examination and treatment device

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIMENS AKTIENGESELLSCHAFT

(21) Appl. No.: 12/380,788

(22) Filed: Mar. 3, 2009

(30) Foreign Application Priority Data
Mar. 12, 2008 (DE) 10 2008 013 854.1

(51) Int. Cl. A61M 25/06; A61B 6/00; A61B 5/055; A61B 8/12

(52) U.S. Cl. 600/411; 600/424; 604/523; 604/508

(57) ABSTRACT

A catheter for treatment of the heart with a flexible catheter sheath surrounding a catheter lumen and with an apparatus for implanting cell material, which comprises an injection apparatus arranged in an area of the catheter tip, is to be created such that the risk of such an intervention is reduced in relation to previously known and practiced concepts. To this end there is inventive provision for at least one imaging sensor to be arranged in the area of the catheter tip.

### CROSS REFERENCE TO RELATED APPLICATIONS

This application claims priority of German application No. 10 2008 013 854.1 filed Mar. 12, 2008, which is incorporated by reference herein in its entirety.

### FIELD OF THE INVENTION

The invention relates to a catheter for cardiac treatment with a flexible catheter sheath surrounding a hollow catheter lumen with an apparatus for implantation of cell material which includes an injection facility arranged in the area of the catheter tip.

The invention also relates to a medical examination and treatment device with such a catheter.

### BACKGROUND OF THE INVENTION

Among the most frequent diseases with fatal outcomes are vascular diseases with consequent illnesses such as coronary infarctions or strokes. The coronary infarction is caused by a disease of the coronary vessels. In such cases arteriosclerotic deposits (plaque) result in the formation of a local thrombus which can lead to a total blockage (occlusion) of coronary vessels and thus to the blood flow being blocked. The occlusion in a coronary infarction is currently treated in the majority of cases by what is known as a PCTA (Percutaneous Transluminal Coronal Balloon Angioplasty). In these cases the constrictions of the coronary vessels are expanded with the aid of a catheter-guided balloon. However this treatment does not allow already dead (necrotic) heart muscle tissue to regenerate again.

Since 2001 first experiments have been performed on living beings to regenerate dead tissue in the heart. In such cases what is referred to as myogenesis has been established over time. This is a method in which body cells, especially stem cells, are injected directly into the heart muscle in the area of the infarction cicatrix. The stem cells form new muscle cells there which improve the pumping function of the heart. The first attempts were initially made in each case within the course of a surgical intervention in open heart surgery. Since this very intensive intervention, in which inter alia a heart-lung machine is used, has significant risks associated with it, the minimally-invasive injection of stem cells into the heart muscle with catheters embodied specifically for the purpose and injection needles attached thereto has become established.

In addition, what is referred to as angiogenesis is also known, in which the coronary vessels are “flushed through” with a comparatively large volume of a solution containing body cells, for which purpose however a relatively large quantity of body cells must be produced in bioreactors.

In the minimally-invasive method of operation a thin flexible hollow body or catheter is introduced from the groin or from the arm of the patient starting in the blood vessel (veins or arteries) and pushed forwards until such time as the (proximal) end of the catheter facing the body—the catheter tip—reaches the area of the heart to be treated. Arranged in the area of the catheter tip is an injection facility, with which the body cells are introduced into the coronary region concerned. After the treatment has been undertaken the catheter is withdrawn again via the blood vessel and thus removed.

Since the availability and usability of a few types of stem cell, for example embryonic or fetal stem cells, is restricted for various reasons, myogenesis is becoming ever more established with so-called satellite cells (skeletal myoblasts).

Such satellite cells exist as precursor cells, which lie in an idle state under the basal membrane of the muscle fibers. If the skeletal muscle is injured the cell cycle is activated in these cells and these cells begin to divide and change into functional muscle cells which heal the injured skeletal muscle. Satellite cells are frequently taken from the upper thigh for myogenesis and reproduced in a bioreactor and then injected into the patient with the aid of an injection catheter into the heart muscle.

Such an injection catheter is for example known from US 2004/0010231.

Although the minimally-invasive method of operation represents a significant advance compared to surgical intervention on an open heart, an intervention with which an implant of cell material in the heart is undertaken with the aid of a catheter still represents a risk for the patient which is not to be underestimated.

### SUMMARY OF THE INVENTION

The object of the present invention is thus to specify a catheter and an associated medical examination and treatment device with which the risk of such an intervention can be further reduced compared to previously known and practiced concepts, and the likelihood of a comprehensive successful treatment can be increased.

In relation to the catheter the object is inventively achieved by at least one imaging sensor being arranged in the area of the tip of the catheter.

Expediently the catheter is a component of a medical examination and treatment device, with the imaging sensor being connected by a signal line routed in the lumen of the catheter to an image editing and reproduction device located outside the catheter and transmitting image information in real time to the latter from the location of an intervention.

The invention is based on the idea that a major disadvantage of previous body cell injection catheters and their handling lies in their being applied to the heart with the aid of external x-ray illumination (angiography), so that the patient and the medical personnel are subjected to ionizing radiation during this procedure. This represents a danger to the health of the patient, but especially also for the medical personnel who regularly conduct these types of intervention, which is to be avoided if at all possible. Added to this is the risk of the cells being able to be damaged or changed by the ionizing radiation during an intervention for injection of cell material into the heart.

A further disadvantage lies in the fact that in the x-ray image either the catheter and/or the local environment of the catheter within the body are comparatively difficult to see, especially when a low-cost conventional x-ray method with two-dimensional imaging characteristics is used. Although injection of contrast media can show the immediate vicinity of the catheter tip more clearly and give better contrast, there are also patients who have allergic reactions to contrast media which can lead to dangerous complications. The restricted resolution of the presentation with angiographic x-ray fluoroscopy also runs the risk of the location of the injection tool not being able to be sufficiently accurately checked during the intervention, and thereby of the injection tool not being able to be positioned sufficiently accurately.

For avoiding such difficulties there is now provision for arranging an imaging sensor in the vicinity of the injection facility or of the injection tool. This allows a comparatively precise and high-resolution presentation of the spatial environment of the injection tool. With the imaging sensor “live images” can be transmitted from the location of the minimally-invasive intervention, i.e. directly from the heart, to an eternally-located reproduction device, e.g. a computer-controlled visualization system with connected monitor. The insertion and movement of the catheter through the vessel, heart chambers and heart valves and the precise positioning of the injection tool can be followed under real time control. The high-resolution presentation of the position made possible by this allows fine corrections to the position of the catheter to be made right away. In particular the risk of an avoidable “sticking” of the injection tool into areas of the body tissue not intended for this, for example in the vessel walls during the guidance of the catheter through a blood vessel, can be reduced.

Thus an application of x-ray radiation during the intervention can at least be largely reduced. If required an x-ray image can still be recorded at selected points in time as a control to supplement the imaging with the aid of the catheter.

Advantageously the imaging sensor is configured and aligned so that the field of vision covers a spatial area around the injection tool. This means that the imaging sensor—in relation to the catheter sheath arranged approximately cylindrically around center axis essentially “looks” radially outwards, depending on the specific arrangement and/or on the type and functional principle of the sensor and/or depending on the material of the injection tool, if necessary also “through” the injection tool or past it.

In an alternate embodiment there is provision for the field of vision of the imaging sensor to primarily cover the spatial area lying in front of the catheter tip, i.e. looking “forwards” in relation to the direction of insertion of the catheter, which is especially useful during the injection process, as well as for monitoring the insertion process of the catheter and its advance, e.g. through a heart valve.

Optimally the two above-mentioned options are combined with each other in a suitable manner for the imaging sensor so that the sensor has a field of vision both in the radial direction and also in the forwards direction that is as large as possible. Alternatively, provided there is sufficient space available, a number of imaging elements or sensors can also be provided, which complement each other by covering different angular areas.

Advantageously the imaging sensor is able to be moved in a longitudinal direction in relation to the outer catheter sheath. For example there can be provision for moving the sensor out from a “withdrawn” position in the vicinity of the proximal end of the outer catheter sheath in a forwards direction out of the catheter sheath, in order, while holding the catheter sheath in a constant position, to define a variably positionable observation point from which the areas lying further forwards can be inspected. For this purpose the imaging sensor can be arranged for example on an inner catheter able to be moved relative to the outer catheter sheath and in its lumen or on an inner part.

Expediently the injection tool comprises an injection needle through which cell material can be injected locally from an appropriate reservoir or storage container into the heart tissue. The storage container is expediently arranged outside the patient's body. The stem cells held in a solution for example are supplied to the injection needle in this case via a supply line running in the hollow section of the catheter and connected at the distal end of the catheter via a coupling piece to the storage container. The on-demand transport of the solution through guidance system can be undertaken in such cases for example by a pressurized propellant fluid or with the aid of a pump drive. Alternatively a smaller stock of cells could also be held in a local reservoir within the catheter sheath and in a similar way to an injection needle be pushed into a hollow needle (tube) by displacement of a piston.

In a preferred embodiment the injection needle is able be adjusted variably in relation to the catheter in its longitudinal position, especially from a completely withdrawn initial position to a completely extended end position, and vice versa. This can be done for example by an electronic or mechanically activated drive unit in the inside of the catheter. Alternatively there can be provision for actuation via an actuation element guided in the catheter lumen, e.g. a wire able to be moved in a longitudinal direction. With a withdrawn injection needle the risk of the needle sticking into a vessel during the navigation of the catheter into the target area can especially be reduced. On reaching the target area the injection needle can then be deployed and positioned, and this can be done using real time monitoring with the aid of the imaging sensor.

Preferably the imaging sensor is implemented as an (acoustic) ultrasound sensor, as a magnetic resonance sensor or as an optical imaging sensor.

Imaging with ultrasound (sonography) is undertaken using the so called echo-impulse method. An electrical impulse of a high frequency generator is converted in the sound head of an ultrasound converter (mostly a piezo crystal, possibly also a silicon-based sensor) into a sound pulse and transmitted. The sound wave is partly or completely scattered or reflected on the inhomogenities of the tissue structure. A returning echo is converted in the sound head into an electrical signal and then visualized in a connected electronic evaluation and display unit, with a 2D or 3D scan of the area under examination able to be performed by a mechanical or electronic pivoting of the sensor. Intervascular ultrasound imaging (IVUS) is especially suitable for imaging of deeper layers of tissue and vessel structures.

In a second advantageous variant the imaging sensor involves a so-called IVMRI sensor for intervascular magnetic resonance tomography (MRI=Intra Vascular Magnetic Resonance Imaging). In magnetic resonance tomography the magnetic moments (magnetic resonance) of the atomic nuclei of the tissue to be examined are aligned in an external magnetic field and excited by introduced radio waves to move in an orbit (precession), with as a result of relaxation processes in an assigned receive coil, an electrical magnetic resonance signal being induced which represents the basis for the image calculation.

Recently there has been success in miniaturizing the elements generating the magnetic fields as well as the transmit and receive coils and integrating them into an imaging IVMRI sensor such that an intracorporal or intervascular application of the MRI method (MRI=Magnetic Resonance Imaging) is possible, with advantageously the requested static magnetic field being created or applied within the body of the patient. Such a concept is described for example in U.S. Pat. No. 6,600,319.

For this purpose a permanent magnet or an electromagnet for creating a static magnetic field and a coil equally effective as a transmit and receive coil are integrated into the IVMRI sensor. The magnet creates field gradients of preferably 2 T/m through 150 T/m in the vicinity of the vessel or organ under examination. In the vicinity means in this case up to 20 mm away from the magnet. Depending on the strength of the magnetic field, radio waves in the frequency range of 2 MHz through 250 MHz can be coupled out via the coil for exciting the surrounding body tissue. Higher static magnetic field strengths demand higher frequencies in the excitation field. The coil advantageously also serves for receiving the associated “response field” from the body tissue. In an alternate embodiment separate transmit and receive coils can be provided.

By contrast with conventional MRI systems, the WMRI sensor and the electronic circuits and digital evaluation unit provided for signal processing and evaluation are advantageously designed so that they also operate with a comparatively inhomogeneous magnetic field with high local field gradients and can create corresponding magnetic resonance images. Since under these conditions the received echo signals are influenced in a characteristic way by the microscopic diffusion of water molecules in the tissue under examination, as a rule an outstanding presentation and differentiation between different soft tissue parts, e.g. between lipid layers and fibrous tissue, is made possible. It is precisely in the area of application of minimally-invasive interventions that this is of especial interest. It is namely known from recent investigations that marked stem cells in particular and also the typical infarction regions in the heart can be displayed well using MRI.

As an alternative to the concepts described here, the static magnetic field can also be created by external magnets. By contrast with conventional MRI, the dynamic fields, i.e. the radio waves are created, but also within this embodiment expediently intervascular, i.e. by a number of send and receive units arranged on the catheter.

Furthermore, in an alternate or additional embodiment, an optical imaging element can be provided in the area of the catheter tip. For example an optical semiconductor detector for detecting incident light based on the known CMOS technology (CMOS=Complementary Metal Oxide Semiconductor) is considered. Such a CMOS sensor, also known as an “Active Pixel Sensor” is based, in a similar manner to the CCD sensors (CCD=Charge-Coupled Device) primarily known from the field of digital photography on the internal photoelectric effect and, as a well as a low power consumption, has the advantage of being especially cost-effective to produce. For illuminating the examination and treatment region, with this variant of the imaging a suitable light source, e.g. an LED (LED=Light Emitting Diode) is to be provided in the area of the catheter tip, which can be supplied with electrical power via an electrical line routed through the catheter lumen.

In a further embodiment variant the catheter can also be equipped with a sensor for Optical Coherence Tomography (OCT).

Optical coherence tomogaphy imaging delivers high-resolution images, which especially reproduce the structures in the vicinity of the vessel surface comparatively precisely. The principle of this method is based on the fact that light conveyed by the catheter over an optical fiber, preferably infrared light, is beamed into the vessel or onto a tissue structure, with the light reflected there being coupled back into the optical fiber and fed to an evaluation device. In the evaluation unit—in a similar manner to a Michelson interferometer—the interference of the reflected light with the reference light is evaluated for image creation.

While conventional interferometric apparatus preferably operates with laser light of a defined wavelength, which possesses a comparatively large optical coherence length, the so-called LCI (LCI=Low Coherence Interferometry) employs light sources with wideband radiation characteristics (“white light”) and with comparatively low coherence length of the emitted light. Corresponding image sensors which are now provided in accordance with an advantageous embodiment of the invention for use in the catheter, are for example described in US 2006/0103850.

In an advantageous variation an image sensor can also be provided which is based on the so-called OFDI (OFDI=Optical Frequency Domain Imaging) principle. The method is related to OCT but employs a wider frequency band. The functional principle is described in more detail for example in the publication entitled “Optical frequency domain imaging with a rapidly swept laser in the 815-870 nm range”, H. Lim Et al., Optics Express 5937, Vol. 14, No. 13.

Finally the catheter can also feature an imaging sensor which is based on so-called “Near-Infrared (NIR) Diffuse Reflectance Spectroscopy”.

Furthermore combinations of at least two optical sensors of the above-mentioned type can also be present.

A tabular overview summarizes the strengths and weakness of the respective optical imaging methods (of ++=especially good or suitable through to −−=deficient or unsuitable):

| Comparison of | | | |
|---|---|---|---|
| the image sensors | Near resolution | Far resolution | Penetration of blood |
| Optical (CMOS) | + | + | − |
| OCT | ++ | − | −− |
| LCI | + | + | + |
| NIR | − | − | +/− |
| OFDI | ++ | − | + |

Since the spatial angle able to be detected or viewed with the respective imaging sensor is usually restricted, it is especially advantageous for the configuration with a radial direction of view already mentioned (in relation to the center axis of the catheter) for the imaging sensor to be supported rotatably via a drive shaft guided in the catheter lumen in relation to the outer catheter sheath and in relation to the injection tool. This makes it possible, without the outer catheter sheath itself having to turn in relation to the environment within the body, to obtain a 360° circular view.

Alternatively it is also conceivable to distribute a plurality of imaging sensors around the circumference of the catheter and preferably looking outwards and to provide a cyclic data readout from the sensors e.g. via a multiplexer. Such a configuration is for example implemented by the sensors being arranged fixed on the catheter sheath. As an alternative (or in addition) to this the (or additional) sensors can also be arranged within the catheter sheath grouped around the injection needle. Advantageously they are able to be moved longitudinally—if necessary as sensor clusters or separately. With this type of configuration only a single signal line is required within the catheter sheath, via which the image data of the different sensors is sent or interrogated in turn in the manner of a serial interface. A small number of signal lines, preferably only a single line, limits the space required within the catheter sheath and is thus of advantage for the usability of the mechanical flexibility and bendability of the catheter sheath.

The (mechanical or electronic) rotation of the image sensor during simultaneous withdrawal or advance, using suitable methods of signal processing and image computation principally known from the prior art, advantageously enables 3D images or volume data sets to be created.

In an advantageous development a number of position sensors or position generators are arranged in the area of the catheter tip, by means of which the current position and preferably also the orientation of the catheter tip or the injection needle can be determined. The position sensor or each position sensor in this case is expediently arranged on the outer catheter sheath and/or on the injection tool. Preferably the position sensor or each position sensor comprises a number of electromagnetic transmit coils which interact with a number of external receive coils or signal detectors arranged outside the patient.

In an alternate embodiment the role of the transmit and receive units can also be reversed; meaning that the receive coils are fixed on the catheter side while the transmit coils are preferably arranged as stationary coils in the room.

In a further expedient embodiment a number of passive sensors are fixed on the catheter side, for example a number of RFID (RFID=Radio Frequency IDentification) transponders. A response signal is induced in an RFID transponder from a signal sent out by a stationary transmit coil, which is received by a stationary receive coil and allows a precise spatial localization of the RFID transponder. A passive sensor thus does not need any external energy supply, and also advantageously no supply lead from outside.

The position information received from the position sensor or from each position sensor makes it easier on the one hand to safely introduce the catheter and navigate it to the target area, on the other hand they advantageously support the reconstruction of three-dimensional images from a plurality of two-dimensional cross-sectional images. In addition the position data is advantageously able to be included in the computational correction of movement artifacts and the like.

In a further expedient embodiment at least one magnetic element for guiding the catheter by means of an external magnetic field can be provided in the area of the catheter tip. With this so-called magnetic navigation the catheter will be controlled and driven by an external magnetic field. The respective magnetic element can involve a permanent magnet or an electromagnet.

Mechanical navigation can be provided as an alternative to guiding the catheter by an external magnetic field. Suitable mechanical elements, e.g. in the form of pull wires and the like, are expediently integrated into the catheter for this purpose which, through external tensile or compressive forces, allow a temporary mechanical deformation, expansion and/or bending of the catheter or of individual selectable catheter sections, especially of the catheter tip. Preferably the catheter is automatically guided mechanically and/or magnetically with the aid of a computer-based control and drive device.

There can also be provision for the actual injection catheter to be guided through an outer guide catheter up to the organ to be treated. For example, after an injection of stem cells has been performed in a local area of the heart with a catheter of the type described above, that catheter can be replaced within the guide catheter by a further catheter with the aid of which a further step in the treatment sequence is undertaken in the heart, without any strain being imposed on the patient by renewed invasion, not to mention a change or a movement or other manipulation of the outer guide catheter. The replaced inner catheter also does not first have to be laboriously navigated into the target area and adjusted there again. Instead it is sufficient to insert it up to a stop position in the lumen of the outer guide catheter which remains during the procedure in its previously reached or assumed position in the vessel or in the heart.

An expedient workflow for the used of a body cell injection catheter with integrated imaging typically appears as set down below:
1. Positioning of the patient on the treatment table,
2. Possible preparatory x-ray examination and/or extracorporal ultrassound examination,
3. Introduction of the catheter via a vein access,
4. Guidance of the catheter based on the integrated imaging up to the region of the heart to be treated,
5. Observation of the heart tissue to be treated and positioning of the catheter with the aid of the integrated imaging, especially orientation of the injection tool with the aid of the integrated imaging.
6. Performing an injection of cell material using real time observation by means of the integrated imaging.
7. Removal of the catheter,
8. Possible repetition of steps 3 to 5 with a further catheter and possibly execution of subsequent treatment steps,
9. Possible supplementary final x-ray checking examination and/or extracorporal ultrasound examination,
10. Moving the patient.

Depending on the type of imaging and its capability for “penetration” of blood, it can be sensible during steps No. 4 to No. 6, to flush out the area to be observed from time to time with an electrophysiological saline solution, in order in a one-off operation for a brief period or in brief pulses in periodically repeated cycles to force out or thin the blood. In addition it can be sensible to apply a contrast medium at the location of the observation, based on gadolinium in the case of IVMRI imaging for example, or based on a sulfur hexane fluoride for ultrasound imaging. Advantageously the injection is undertaken via an injection line laid in the catheter lumen or the like, featuring an outlet opening in the area of the catheter tip.

In summary the catheter described here above all makes possible an optimization of the medical operating sequences for a minimally-invasive intervention in the heart of a living being in which an injection of cell material can be conducted. These types of interventions can be completed with a higher degree of patient safety and at the same time more quickly than previously.

### BRIEF DESCRIPTION OF THE DRAWINGS

Different exemplary embodiments of the invention are explained in greater detail below with reference to a drawing. The figures show greatly simplified and schematic diagrams in each case, as follows:

FIG. 1 a medical examination and treatment device with a catheter shown in longitudinal cross section for injection of body cells into an organ of the body, especially in a coronary vessel or a heart muscle,

FIG. 2 to 5 alternate embodiments of such a catheter,

FIG. 6 a detailed diagram of an optical sensor arranged to the side of the injection needle of a catheter of the aforementioned type with a lateral/radial direction of observation,

FIG. 7 a detailed diagram of an optical sensor with its direction of observation pointing forwards,

FIG. 8 a detailed diagram of a sensor head for OCT or LCI imaging with a lateral/radial direction of observation,

FIG. 9 a detailed diagram of a sensor head for OCT or LCI imaging with a direction of observation pointing forwards,

FIG. 10 a detailed diagram of a sensor for IVMRI imaging with a lateral/radial direction of observation, and

FIG. 11 a detailed diagram of a sensor for IVMRI imaging with a forwards direction of observation.

The same parts are shown by the same reference symbols in all the figures.

### DETAILED DESCRIPTION OF THE INVENTION

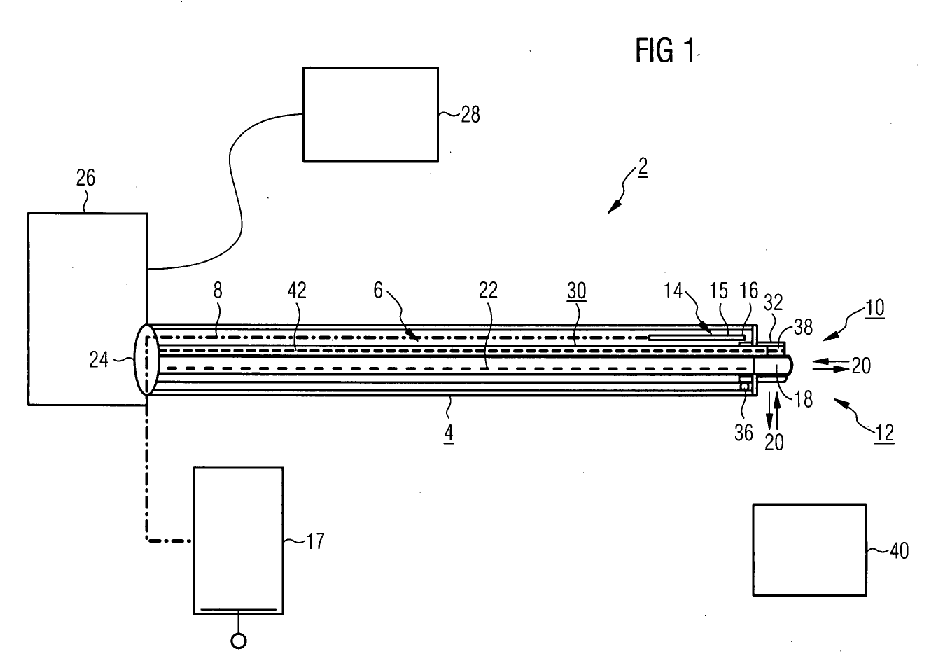

The catheter 2 shown in FIG. 1 is designed for a minimally-invasive surgical intervention in the heart. It comprises a flexible catheter sheath 4 for introduction into a blood vessel not shown in any greater detail. To conduct the intervention, the catheter tip 12 located at the proximal end 10 is pushed forwards up to the treatment region in the heart. The catheter sheath 4 surrounds a cylindrical lumen in the catheter 6 (also referred to as the lumen),within which run lines such as a control line not shown here or a control wire for activation of an apparatus 14 for implantation of cell material in the heart. This apparatus 14 comprises an injection tool 15, which is embodied here in the form of an injection needle 16.

With the aid of the injection needle 16 cell material, especially stem cells held in a solution, can be injected from a reservoir 17 located outside the catheter 2 locally into the heart tissue. The solution in such cases is fed for example with the aid of a propellant fluid under pressure or with a pump apparatus not shown here through the feed line 8 running in the catheter lumen 6 to the injection needle 16. FIG. 1 shows the injection needle 16 in a transport position completely withdrawn or pulled back into the catheter sheath 4. To carry out the injection the injection needle 16 is moved in a proximal direction out of the catheter sheath 4 and thus brought into the handling position. The feed line 8 for the body cells to be injected can for example also be integrated as a lumen in the control wire for the movement of the injection needle 16. Alternatively the two components can be embodied separately from each other.

For an optimum and lasting successful healing and for minimizing possible intervention risks it is important for the catheter 2 and its local environment in the inside of the body to be able to be observed at the best possible resolution during its advance through a blood vessel to the heart for timely and fine corrections to its position. It is important in particular for the injection needle 16 to be positioned as exactly as possible at the right or “appropriate” point on the heart muscle tissue for the respective intervention. This type of monitoring has previously usually been undertaken by angiographic x-ray checking.

For a qualitatively improved monitoring without use of ionizing x-ray radiation the catheter 2 in accordance with FIG. 1 is now equipped with an imaging sensor 18, which is arranged to the side of the injection needle 16 in the area of the catheter tip 12. The “field of vision” of the sensor 18, depending on the sensor and other details of the embodiment, is preferably directed radially outwards (towards the surrounding vessel wall) and/or in a proximal direction forwards (i.e. in the direction of advance of the catheter 2), as is indicated symbolically by the arrows 20.

The imaging sensor 18 can for example be an optical sensor, an acoustic (ultrasound) sensor or a sensor based on the principle of magnetic resonance. The signal and power supply lines 22 needed for its operation and for transmission of the image data that it records are routed in the interior of the catheter sheath 4 up to a connection coupling 24 arranged at the (distal) end of the catheter 2 facing the body. The connection coupling 24 one the one hand allows mechanical connection of the lines carrying compressed air and/or fluid, especially the feed line 8 for the biological cell material to be injected, within the catheter sheath 4 to the external storage container and the like. On the other hand the imaging electronic components of the catheter 2 are electrically connected via the connection coupling 24 to an only schematically indicated signal interface 26, which for its part is connected to an external image processing and reproduction device 28. A monitor not shown in any greater detail is used for reproduction of “live images” recorded intervascularly or intracorporally by the imaging sensor 18 and if necessary subsequently computer-edited from the treatment area.

To enable the imaging sensor 18 to be rotated within the stationary catheter sheath 4 around its own axis, a rotatable drive shaft can be arranged in the catheter lumen 6, which is likewise not shown in any further detail in FIG. 1. The imaging sensor 18, the signal lines 22 and if necessary the drive shaft can be grouped together into a compact unit in the form of an inner catheter arranged within the outer catheter sheath 4 and be surrounded by an (internal) protective sheath 30. During application of interferometric imaging methods in particular optical fibers can also be laid in the inner catheter, via which incident and reflected light bundles are fed to an eternally-sited interferometer unit connectable via the connection coupling 24 or the like. In the area of the imaging sensor 18 the internal protective sleeve 30 and/or the outer catheter sheath 4 and/or the injection tool 15, expediently features a transparent area, if necessary also an optical lens transparent for the respective imaging method.

In addition one or more (optional) lines (not shown here) for a flushing fluid or a contrast medium can be provided, which is able to be injected via an outlet opening 36 located in the vicinity of the imaging sensor 18 at the proximal end of the catheter sheath 4 into the region of the heart to be examined/to be treated.

Finally in the area of the catheter tip 12, here in FIG. 1 in the immediate vicinity of the imaging sensor 18, position sensors 38 can be provided, which in collaboration with a position detection unit 40 arranged outside the patient's body operating on the transmitter-receiver principle a precise positioning/localization of the catheter tip 12 is made possible by identification of the coordinates of the catheter tip 12. The position data thus obtained can for example be fed to the image processing and reproduction device 28 and can be taken into account in image reconstruction, specifically for artifact correction. The necessary signal lines 42 for the position sensors 38 can likewise be fed within the (inner) protective sleeve 30 essentially in parallel to the signal lines 22 of the imaging sensor 18.

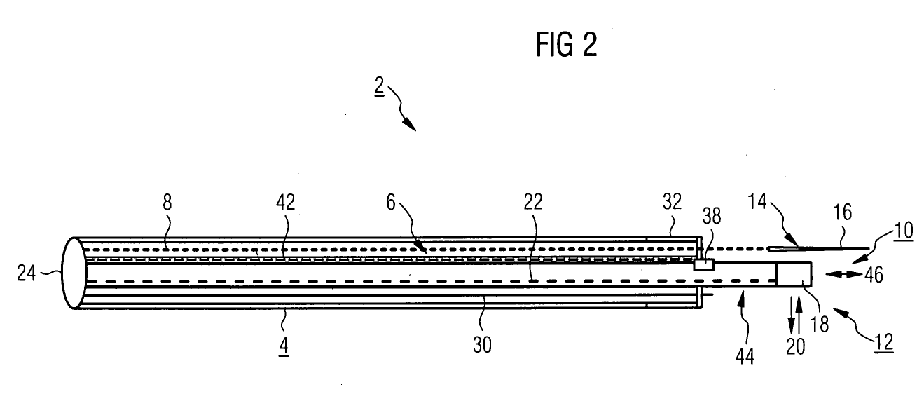

FIG. 2 through FIG. 5 each show constructional variants of the catheter 2.

Thus for example in FIG. 2 the inner section 44 bearing the imaging sensor 18 is moved forwards in relation to the catheter sheath 4 (in the proximal direction) from a withdrawal position not disclosed in any greater detail, corresponding to the position in FIG. 1 into the advanced position shown here and vice versa (indicated by the double arrow 46). This means that the imaging sensor 18 can be pushed forwards if necessary beyond the proximal end of the catheter sheath 4 and has an unrestricted view there, especially of the injection needle 16 likewise moved out of the catheter sheath 4 in FIG. 2. The deployment/withdrawal of the injection needle 16 and of the imaging sensor 18 are preferably possible independently of one another.

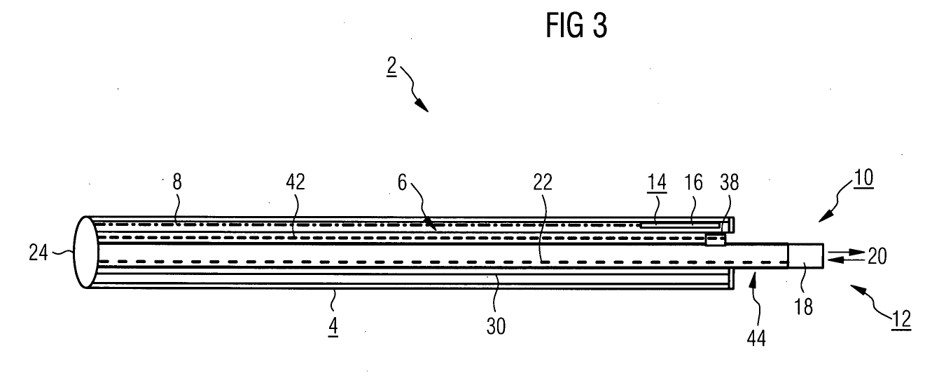

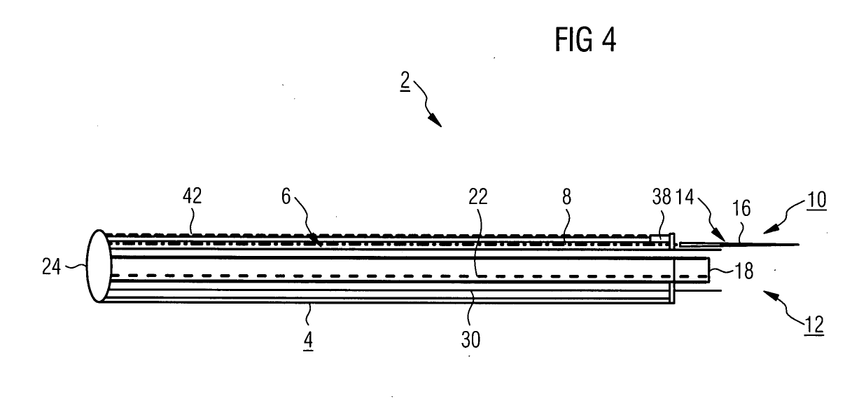

The embodiment in accordance with FIG. 3 essentially corresponds to those depicted in FIG. 1 or FIG. 2, but it dispenses with a transparent window on the catheter sheath 4. The embodiment in accordance with FIG. 4 is also similar to those already described, however position sensor(s) 38 in this variant is/are now arranged on the outer catheter sheath 4. Finally with the variant in accordance with FIG. 5 the movement path of the imaging sensor 18 in the longitudinal direction into the catheter sheath 4 is increased. The position sensors 38 are attached further towards the end of the catheter 2 facing away from the body here and the transparent area 32 is enlarged.

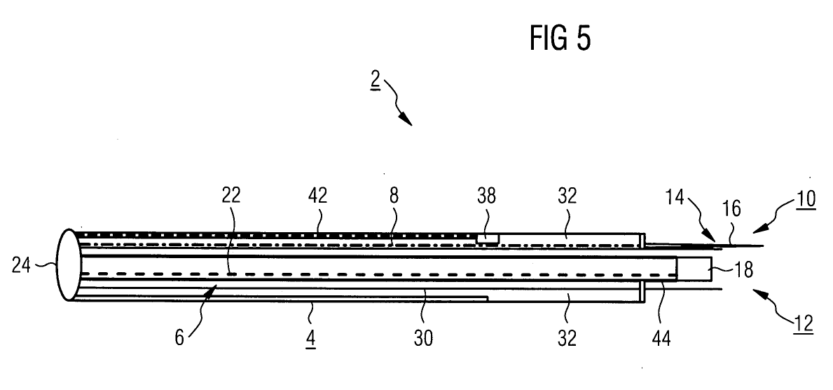

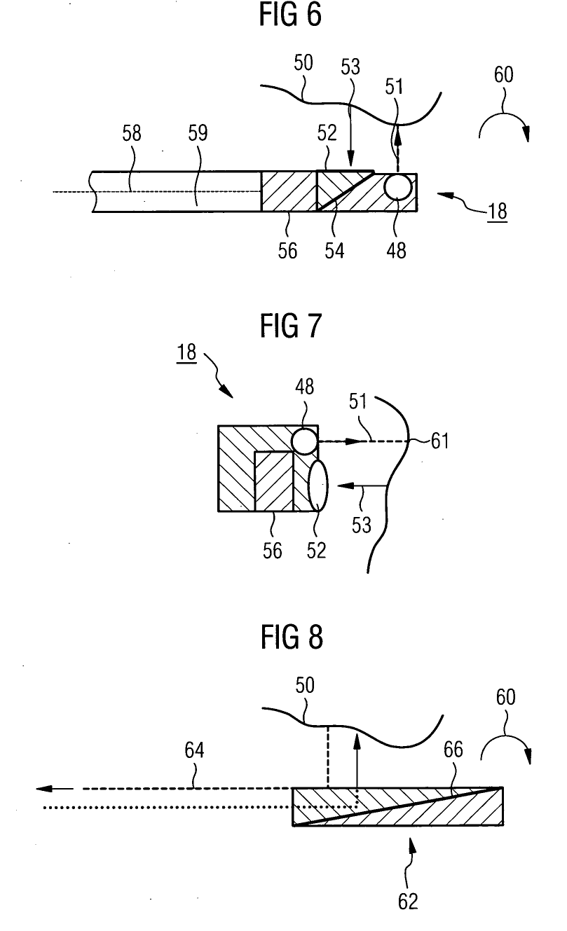

In the detailed diagram shown in FIG. 6 the area of the catheter tip 12 with the imaging sensor 18 is shown enlarged to accentuate it, with a CMOS-based optical sensor being used in the variant shown here. A light source 48, here a high-power micro LED, illuminates the approximately annular vessel wall 50 surrounding the catheter 2 and specifically the imaging sensor 18 (emitted light 51). Light 53 reflected on the vessel wall 50 falls through a lens 52 onto a refection mirror 54 (or also for example onto a prism with a similar method of functioning or beam guidance) and from there onto the actual CMOS image detector 56. The arrangement in accordance with FIG. 6 is also configured for a radial direction of view (relative to the center axis 58 of the catheter 2). A rotational movement effected with the aid of the drive shaft 59 around the center axis 58, indicated by the arrow 60, enables the full lateral 360° field of vision to be covered.

Alternatively FIG. 7 shows an example for a configuration of light source 48, lens 52 and CMOS detector 56 with which a forwards observation is made possible, which is especially useful during the advance of the catheter 2 through the blood vessel up to the heart chamber and if necessary through the hear valve. An obstacle 61 lying in a forwards direction, possibly hindering the further advance, can be detected in this way. The two both variants depicted in FIG. 6 and FIG. 7 can if necessary also be combined with each other in order to provide an especially comprehensive field of vision in practically all directions.

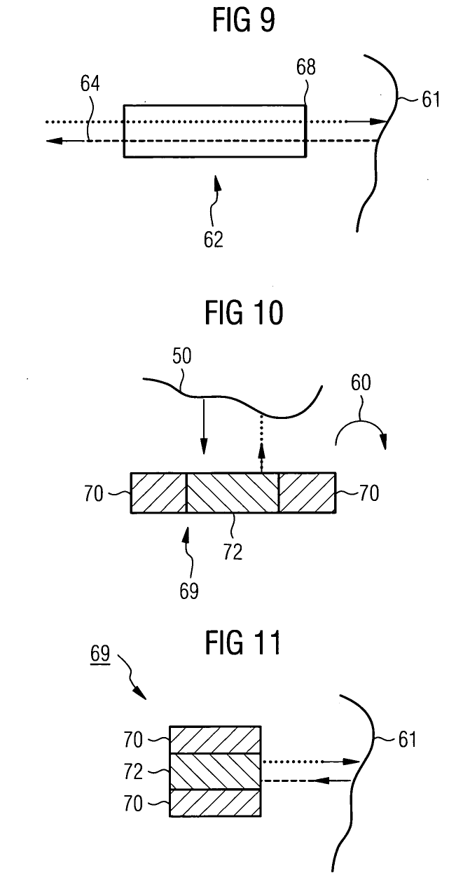

The stated directions of observation, namely in the radial/lateral and forwards directions, can also be implemented with other sensor types. For example FIG. 8 shows a configuration of an OCT or LCI sensor head 62 for radial radiation and reception and FIG. 9 shows it for a forwards radiation and reception. To put it more precisely, the reference symbol 62 only designates the sensor part or the sensor head responsible for coupling light out of and into the optical fiber 64; the actual interferometric evaluation and image generation occurs outside of the catheter 2. Shown in each case is the beam path of coupled out and reflected rays of light influenced by the refection mirror 66 and the lens 68.

In a similar manner an IVMRI sensor or IVUS sensor can also be configured either for radial or forward radiation/reception, as depicted schematically in FIG. 10 and FIG. 11 for an IVMR sensor 69 with permanent magnets 70 for the static magnetic field and transmit/receive coils 72.

With lateral emission/reception it can be advantageous, especially in the case of ultrasound sensors, instead of a single rotating sensor, to provide an array of ultrasound elements with different “directions of view”, which for example are activated, i.e. excited and interrogated cyclically via a multiplexer.

1.-15. (canceled)

16. A catheter for an intervention treatment of a heart of a patient, comprising:
a catheter lumen;
a flexible catheter sheath that surrounds the catheter lumen;
an injection device arranged in an area of a catheter tip that injects a cell material in the heart; and
an imaging sensor arranged in the area of the catheter tip that records an image of the intervention.

17. The catheter as claimed in claim 16, wherein the imaging sensor is aligned so that a field of vision of the imaging sensor covers an area lying all around the injection device.

18. The catheter as claimed in claim 16, wherein the imaging sensor is aligned so that a field of vision of the imaging sensor covers a spatial area lying in a proximal direction in front of the injection device.

19. The catheter as claimed in claim 16, wherein the imaging sensor moves longitudinally relative to the catheter sheath.

20. The catheter as claimed in claim 16, wherein the injection device comprises an injection needle.

21. The catheter as claimed in claim 20, wherein the imaging sensor is arranged laterally to the injection needle.

22. The catheter as claimed in claim 20, wherein a longitudinal position of the injection needle is adjustable relative to the catheter sheath.

23. The catheter as claimed in claim 16, wherein the imaging sensor is an ultrasound element.

24. The catheter as claimed in claim 16, wherein the imaging sensor is a magnetic resonance element.

25. The catheter as claimed in claim 16, wherein the imaging sensor is an optical imaging sensor.

26. The catheter as claimed in claim 16, wherein the imaging sensor rotates relative to the catheter sheath via a drive shaft guided in the catheter lumen.

27. The catheter as claimed in claim 16, wherein a plurality of imaging sensors are distributed around a circumference of the catheter sheath.

28. The catheter as claimed in claim 27, wherein data from the imaging sensors is cyclically readout via a multiplexer.

29. A medical examination and treatment device having a catheter for performing an intervention on a heart of a patient, comprising:
a catheter lumen;
a flexible catheter sheath that surrounds the catheter lumen;
an injection device arranged in an area of a catheter tip that injects a cell material in the heart;
an imaging sensor arranged in the area of the catheter tip that records an image of the intervention;
a signal line that is guided in the catheter lumen; and
an image editing and reproduction device that is located outside the catheter and connected with the image sensor via the signal line,
wherein the imaging sensor is configured to transmit the image from a location of the intervention to the image editing and reproduction device in a real time.

30. A method for minimally-invasive intervention on a heart of a patient by a catheter, comprising:
inserting the catheter through a blood vessel of the patient to a region of the heart to be treated;
arranging an injection device at a proximal end of the catheter in an area of a catheter tip for injecting a cell material in the heart;
arranging an imaging sensor at the proximal end of the catheter in the area of the catheter tip;
recording a real time image of the intervention by the image sensor; and
monitoring an advance of the catheter and controlling a position of the injection device based on the real time image.

31. The method as claimed in claim 30, wherein the injection of the cell material is monitored in a coronary vessel or in a heart tissue of the patient based on the real time image.
